(19) United States
(12) Patent Application Publication
(10) Pub. No.: US 2022/0357308 A1
(43) Pub. Date: Nov. 10, 2022

(54) METHOD AND SYSTEM FOR SELECTING CHEMICAL REAGENTS IN MEASUREMENT OF ASPHALT SURFACE ENERGY

(71) Applicant: Wuhan University of Technology, Wuhan (CN)

(72) Inventors: Rong Luo, Wuhan (CN); Longchang Niu, Wuhan (CN); Chongzhi TU, Wuhan (CN); Jing Luo, Wuhan (CN); Xiang Wang, Wuhan (CN); Qiang Miao, Wuhan (CN)

(21) Appl. No.: 17/863,390

(22) Filed: Jul. 13, 2022

Related U.S. Application Data
(63) Continuation of application No. PCT/CN2021/120076, filed on Sep. 24, 2021.

(30) Foreign Application Priority Data
Jan. 20, 2021 (CN) 202110076963X

(51) Int. Cl. G01N 33/42; G01N 19/04; G06F 17/11

(52) U.S. Cl. CPC G01N 33/42; G01N 19/04; G06F 17/11

(57) ABSTRACT

A method and system for selecting chemical reagents in measurement of asphalt surface energy are provided. The method includes: selecting different chemical reagents and obtaining contact angle values formed between the respective chemical reagents and asphalt slides; obtaining asphalt surface energy parameters corresponding to each of combinations of the chemical reagents according to the contact angle values; obtaining variation coefficients of the asphalt surface energy parameters corresponding to each of the combinations of the chemical reagents and selecting a group of combinations of the chemical reagents according to the variation coefficients; and obtaining numbers of abnormal values of asphalt surface energy components in the group of combinations of the chemical reagents and obtaining a target combination of the chemical reagents according to the numbers of the abnormal values. The combination of the chemical reagents with high stability of testing data can be selected by using the method.

### TECHNICAL FIELD

The disclosure relates to a technical field of a measurement of asphalt surface energy, in particular to a method and system for selecting chemical reagents in the measurement of asphalt surface energy.

### DESCRIPTION OF RELATED ART

As one of main structural forms of highways and urban roads in China, asphalt pavements are more and more widely used. During design and construction of surface layers of the asphalt pavements, an important factor that directly affects pavement performance of asphalt mixtures, such as water stability, self-healing ability and fatigue cracking life, is an adhesion performance between asphalt and aggregate.

In order to evaluate the adhesion performance between the asphalt and the aggregate with the help of detailed and accurate testing data. A surface free energy method is used internationally to determine a size of the adhesion performance which is used as a quantitative index. A primary task of determining the quantitative index is to accurately measure surface energy parameters of the respective asphalt and the aggregate, and then solve it with the help of an equation provided by a surface energy theory system. At present, a Good-van Oss-Chaudhury (GvOC) surface energy theory system is widely used in the pavement industry at home and abroad. The system stipulates that the asphalt and the aggregate respectively have three basic surface energy parameters, including a non-polar component, a polar acid component and a polar alkali component.

For asphalt materials, most common testing methods for determining the three surface energy parameters are an inserting plate method (also referred to as Wilhelmy plate method) and a static drop method. Under the existing surface free energy theoretical system and testing conditions, different chemical reagents are selected and the same testing method is used for the test. Because the simultaneous equations are required in the process of solving asphalt surface energy parameters, therefore, at least three different chemical reagents with known surface energy parameters are selected for random combination. However, there are obvious differences in the asphalt surface energy parameters of same asphalt measured by different combinations of the chemical reagents, and solved results of some combinations of the chemical reagents will even have negative values, resulting in the inability to solve. Therefore, in the face of multiple groups of testing data with obvious differences, how to select one combination of the chemical reagents with high stability of testing data and how to formulate a reasonable and effective data stability evaluation scheme in the measurement of asphalt surface energy have undoubtedly become important problems to be solved.

### SUMMARY OF THE DISCLOSURE

In view of this, it is necessary to provide a method and system for selecting chemical reagents in measurement of asphalt surface energy, so as to solve the problem that the one combination of the chemical reagents with high stability of testing data cannot be selected in the prior art.

Specifically, an embodiment of the disclosure provides a method for selecting chemical reagents in the measurement of asphalt surface energy, including:

selecting different chemical reagents and obtaining contact angle values formed between the respective chemical reagents and asphalt slides;

obtaining asphalt surface energy parameters corresponding to each of combinations of the chemical reagent according to the contact angle values formed between the respective chemical reagents and the asphalt slides;

obtaining variation coefficients of the asphalt surface energy parameters corresponding to each of the combinations of the chemical reagents and selecting a group of combinations of the chemical reagents from the combinations of the chemical reagents according to the variation coefficients of the asphalt surface energy parameters corresponding to each of the combinations of the chemical reagents;

obtaining numbers of abnormal values of asphalt surface energy components (also referred to as asphalt surface energy parameters) in the group of combinations of the chemical reagents and obtaining a target combination of the chemical reagents from the group of combinations of the chemical reagents according to the numbers of the abnormal values of asphalt surface energy components.

In an embodiment, the obtaining contact angle values formed between the respective chemical reagents and asphalt slides, includes:

calculating variation coefficients of the respective contact angle values formed between the respective chemical reagents and asphalt slides under different testing methods to obtain one testing method with minimum data discrete degree from the different testing methods, and obtaining the contact angle values formed between the respective chemical reagents and the asphalt slides under the one testing method with minimum data discrete degree.

In an embodiment, the obtaining asphalt surface energy parameters corresponding to each of combinations of the chemical reagents according to the contact angle values formed between the respective chemical reagents and the asphalt slides, includes:

obtaining multiple values of the asphalt surface energy parameters corresponding to each of the combinations of the chemical reagents according to the contact angle values formed between the respective chemical reagents and the asphalt slides and obtaining the asphalt surface energy parameters corresponding to each of the combinations of the chemical reagents according to the multiple values of the asphalt surface energy parameters and by concurrently taking a minimum fitting error of the asphalt surface energy parameters as a target value.

In an embodiment, the obtaining multiple values of the asphalt surface energy parameters corresponding to each of the combinations of the chemical reagents according to the contact angle values formed between the respective chemical reagents and the asphalt slides, includes:

obtaining the multiple values of the asphalt surface energy parameters corresponding to each of the combinations of the chemical reagents according to the contact angle values formed between the respective chemical reagents and the asphalt slides and a calculation formula of asphalt surface energy parameters; and the calculation formula of asphalt surface energy parameters is:

$$\sqrt{{γ}_{S}^{-} {γ}_{L}^{+}} + \sqrt{{γ}_{S}^{+} {γ}_{L}^{-}} + \sqrt{{γ}_{S}^{L w} {γ}_{L}^{L w}} = \frac{( 1 + cos θ ) {γ}_{L}}{2} ,$$

where γ_{S}^{Lw} is a nonpolar component of asphalt surface energy, γ_{L}^{Lw} is a nonpolar component of chemical reagent surface energy, γ_{S}⁻ is a polar alkali component of asphalt surface energy, γ_{S}⁺ is a polar acid component of asphalt surface energy, γ_{L}⁻ is a polar alkali component of chemical reagent surface energy, γ_{L}⁺ is a polar acid component of chemical reagent surface energy, γ_{L} is a total energy of chemical reagent surface energy, and θ is a contact angle.

In an embodiment, the obtaining the asphalt surface energy parameters corresponding to each of the combinations of the chemical reagents according to the multiple values of the asphalt surface energy parameters and by concurrently taking a minimum fitting error of the asphalt surface energy parameters as a target value, includes:

obtaining the asphalt surface energy parameters corresponding to each of the chemical reagent combinations according to multiple values of each of the asphalt surface energy parameters and by concurrently taking the minimum fitting error as the target value; and the minimum fitting error is calculated as that:

$$Min = \frac{\vert ( \sqrt{{γ}_{S}^{L W} {γ}_{L}^{L W}} + \sqrt{{γ}_{S}^{+} {γ}_{L}^{-}} + \sqrt{{γ}_{S}^{-} {γ}_{L}^{+}} ) - \frac{{γ}_{L} ( 1 + cos θ )}{2} \vert}{\sqrt{{( \sqrt{{γ}_{L}^{L W}} )}^{2} + {( \sqrt{{γ}_{L}^{-}} )}^{2} + {( \sqrt{{γ}_{L}^{+}} )}^{2}}} .$$

In an embodiment, the obtaining variation coefficients of the asphalt surface energy parameters corresponding to each of the combinations of the chemical reagents and selecting a group of combinations of the chemical reagents from the combinations of the chemical reagents according to the variation coefficients of the asphalt surface energy parameters corresponding to each of the combinations of the chemical reagents, includes:

selecting the combinations of the chemical reagents corresponding to the asphalt surface energy parameters of both two kinds of asphalt being not zero from the different chemical reagents, calculating the variation coefficients of the asphalt surface energy parameters of each of the two kinds of asphalt of the respective combinations of the chemical reagents, and selecting the group of combination of the chemical reagents according to the variation coefficients of the asphalt surface energy parameters of each of the two kinds of asphalt.

In an embodiment, the obtaining numbers of abnormal values of asphalt surface energy components in the group of combinations of the chemical reagents, includes:

obtaining the numbers of the abnormal values of asphalt surface energy components of respective combinations of the chemical reagents in the group of combinations of the chemical reagents according to jump degrees of asphalt surface energy components of the combinations of the chemical reagents corresponding to the asphalt surface energy parameters of both the two kinds of asphalt being not zero.

In an embodiment, the obtaining a target combination of the chemical reagents from the group of combinations of the chemical reagent according to the numbers of the abnormal values of asphalt surface energy components, includes:

taking a combination of the chemical reagents with a least number of the abnormal values of asphalt surface energy components in the group of combinations of the chemical reagents as the target combination of the chemical reagents.

In an embodiment, each of the combinations of the chemical reagents includes three kinds of chemical reagents or four kinds of chemical reagents selected from the different chemical reagents.

An embodiment of the disclosure further provides a system for selecting chemical reagents in measurement of asphalt surface energy, including: a contact angle obtaining module, an asphalt surface energy parameter obtaining module, a chemical reagent combination group obtaining module and a chemical reagent combination determining module;

the contact angle obtaining module is configured to select different chemical reagents and obtain contact angle values formed between the respective chemical reagents and asphalt slides;

the asphalt surface energy parameter obtaining module is configured to obtain asphalt surface energy parameters corresponding to each of combinations of the chemical reagents according to the contact angle values formed between the respective chemical reagents and the asphalt slides;

the chemical reagent combination group obtaining module is configured to obtain variation coefficients of the asphalt surface energy parameters corresponding to each of the combinations of the chemical reagents and select a group of combinations of the chemical reagents from the combinations of the chemical reagents according to the variation coefficients of the asphalt surface energy parameters corresponding to each of the combinations of the chemical reagents; and

the chemical reagent combination determining module is configured to obtain numbers of abnormal values of asphalt surface energy components in the group of combinations of the chemical reagents and obtain a target combination of the chemical reagents from the group of combinations of the chemical reagents according to the numbers of the abnormal values of the asphalt surface energy components; and

the contact angle obtaining module, the asphalt surface energy parameter obtaining module, the chemical reagent combination group obtaining module and the chemical reagent combination determining module are software modules stored in one or more memories and executable by one or more processors coupled to the one or more memories.

Compared with the prior art, the beneficial effects of the disclosure include: the combination of the chemical reagents with high stability of testing data can be selected by selecting the different chemical reagents and obtaining the contact angle values formed between the respective chemical reagents and the asphalt slides, obtaining the asphalt surface energy parameters corresponding to each of the combinations of the chemical reagents according to the contact angle values, obtaining the variation coefficients of the asphalt surface energy parameters corresponding to each of the combinations of the chemical reagents and selecting the group of combinations of the chemical reagents according to the variation coefficients, and obtaining the numbers of the abnormal values of asphalt surface energy components in the group of combinations of the chemical reagents and obtaining the target combination of the chemical reagents according to the numbers of the abnormal values of asphalt surface energy components.

### BRIEF DESCRIPTION OF THE DRAWINGS

FIG. 1 is a flowchart of a method for selecting chemical reagents in measurement of asphalt surface energy according to an embodiment of the disclosure.

FIG. 2 is an image showing an optical contact angle meter according to an embodiment of the disclosure.

FIG. 3 is an image showing a measurement of a contact angle by the static drop method according to an embodiment of the disclosure.

FIG. 4 is an image showing an automatic surface tension meter according to an embodiment of the disclosure.

FIG. 5 is an image showing an Excel calculation table according to an embodiment of the disclosure.

FIG. 6 is an image showing an operation interface of Solver in Excel according to an embodiment of the disclosure.

FIG. 7 is a schematic structural diagram of a system for selecting chemical reagents in measurement of asphalt surface energy according to an embodiment of the disclosure.

### DETAILED DESCRIPTION OF EMBODIMENTS

Embodiments of the disclosure will be described in detail below in conjunction with the accompanying drawings, which form a part of the disclosure and, together with the embodiments of the disclosure, are used to explain principles of the disclosure and are not used to limit the scope of the disclosure.

### Embodiment 1

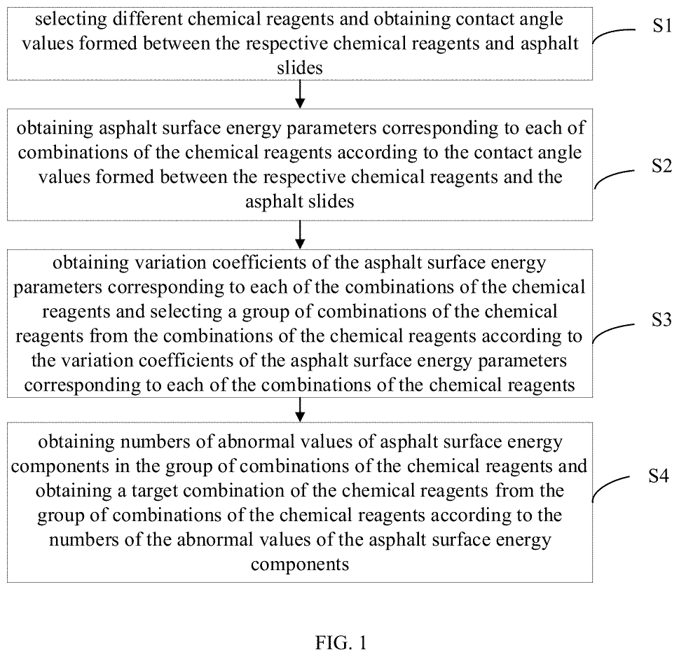

An embodiment of the disclosure provides a method for selecting chemical reagents in measurement of asphalt surface energy. As shown in FIG. 1, the method for selecting chemical reagents in the measurement of asphalt surface energy can include the following steps:

S1, selecting different chemical reagents and obtaining contact angle values formed between the respective chemical reagents and asphalt slides;

S2, obtaining asphalt surface energy parameters corresponding to each of combinations of the chemical reagents according to the contact angle values formed between the respective chemical reagents and the asphalt slides;

S3, obtaining variation coefficients of the asphalt surface energy parameters corresponding to each of the combinations of the chemical reagents and selecting a group of combinations of the chemical reagents from the combinations of the chemical reagents according to the variation coefficients of the asphalt surface energy parameters corresponding to each of the combinations of the chemical reagents; and

S4, obtaining numbers of abnormal values of asphalt surface energy components in the group of combinations of the chemical reagents and obtaining a target combination of the chemical reagents from the group of combinations of the chemical reagents according to the numbers of the abnormal values of asphalt surface energy components.

In a specific embodiment, the asphalt surface energy parameters include a nonpolar component of asphalt surface energy, a polar alkali component of asphalt surface energy, a polar acid component of asphalt surface energy, a polar component of asphalt surface energy and a total energy of asphalt surface energy.

In a specific embodiment, the obtaining asphalt surface energy parameters corresponding to each of combinations of the chemical reagents according to the contact angle values formed between the respective chemical reagents and the asphalt slides, includes:

obtaining multiple values of the asphalt surface energy parameters corresponding to each of the combinations of the chemical reagents according to the contact angle values formed between the respective chemical reagents and the asphalt slides, and obtaining the asphalt surface energy parameters corresponding to each of the combinations of the chemical reagents according to the multiple values of the asphalt surface energy parameters and by concurrently taking a minimum fitting error of the asphalt surface energy parameters as a target value.

In a specific embodiment, the obtaining multiple values of the asphalt surface energy parameters corresponding to each of the combinations of the chemical reagents according to the contact angle values formed between the respective chemical reagents and the asphalt slides, includes:

obtaining the multiple values of the asphalt surface energy parameters corresponding to each of the combinations of the chemical reagents according to the contact angle values formed between the respective chemical reagents and the asphalt slides and a calculation formula of asphalt surface energy parameters, wherein the calculation formula of asphalt surface energy parameters is:

$$\sqrt{{γ}_{S}^{-} {γ}_{L}^{+}} + \sqrt{{γ}_{S}^{+} {γ}_{L}^{-}} + \sqrt{{γ}_{S}^{L w} {γ}_{L}^{L w}} = \frac{( 1 + cos θ ) {γ}_{L}}{2} .$$

where γ_{S}^{Lw} is the nonpolar component of asphalt surface energy, γ_{L}^{Lw} is a nonpolar component of chemical reagent surface energy, γ_{S}⁻ is the polar alkali component of asphalt surface energy, γ_{S}⁺ is the polar acid component of asphalt surface energy, γ_{L}⁻ is a polar alkali component of chemical reagent surface energy, γ_{L}⁺ is a polar acid component of chemical reagent surface energy, γ_{L} is a total energy of chemical reagent surface energy, and θ is a contact angle.

In a specific embodiment, a calculation table of asphalt surface energy parameters is made by using Excel software, and the contact angle values obtained from the test and the surface energy parameter values of the chemical reagent that meet the conditions are putted into the calculation formula of asphalt surface energy parameters and solved with the simultaneous equations. After the nonpolar component of asphalt surface energy γ_{S}^{Lw}, the polar alkali component of asphalt surface energy γ_{S}⁻ and the polar acid component of asphalt surface energy γ_{S}⁺ are obtained, the polar component of asphalt surface energy γ_{S}^{AB} and the total energy of asphalt surface energy γ_{S} are calculated by formulas: γ_{S}^{AB}=2\sqrt{γ_{S}⁺γ_{S}⁻} and Y_{S}=γ_{S}^{AB}+γ_{S}^{LW}.

It should be noted that the non-polar component, the polar alkali component and the polar acid component of chemical reagent surface energy are known, and the contact angle can be obtained by the static drop method and the inserting plate method. According to the contact angle calculation formula, at least three equations can be established to obtain the asphalt surface energy parameters. At the same time, due to the existence of the root sign, each of the asphalt surface energy parameters has multiple values.

In a specific embodiment, the obtaining the asphalt surface energy parameters corresponding to each of the combinations of the chemical reagents according to the multiple values of the asphalt surface energy parameters and by concurrently taking a minimum fitting error of the asphalt surface energy parameters as a target value, includes:

obtaining the asphalt surface energy parameters corresponding to each of the chemical reagent combinations according to multiple values of each of the asphalt surface energy parameters and by concurrently taking the minimum fitting error as the target value, wherein the minimum fitting error is calculated as that:

$$Min = \frac{\vert ( \sqrt{{γ}_{S}^{L W} {γ}_{L}^{L W}} + \sqrt{{γ}_{S}^{+} {γ}_{L}^{-}} + \sqrt{{γ}_{S}^{-} {γ}_{L}^{+}} ) - \frac{{γ}_{L} ( 1 + cos θ )}{2} \vert}{\sqrt{{( \sqrt{{γ}_{L}^{L W}} )}^{2} + {( \sqrt{{γ}_{L}^{-}} )}^{2} + {( \sqrt{{γ}_{L}^{+}} )}^{2}}} .$$

In a specific embodiment, an overall least square method is used to minimize the fitting error to determine the best asphalt surface energy parameters. The fitting error of each equation in the simultaneous equations of the calculation formula of asphalt surface energy parameters is set as the target value. According to the calculation formula of asphalt surface energy parameters and a geometric meaning of the overall least square method, the overall least square method can make the fitting error to the minimum value Min, that is, the shortest straight-line distance from the fitting point to any plane in the spatial rectangular coordinate system.

During a specific implementation, the three asphalt surface energy parameters to be solved are set as variable cells, and an average value of a sum of the three Min values is set as the target value. The results can be calculated by using the function of Solver in Excel.

In a specific embodiment, the obtaining contact angle values formed between the respective chemical reagents and asphalt slides, includes:

calculating variation coefficients of the respective contact angle values formed between the respective chemical reagents and asphalt slides under different testing methods to obtain one testing method with minimum data discrete degree from the different testing methods, and obtaining the contact angle values formed between the respective chemical reagents and the asphalt slides under the one testing method with minimum data discrete degree.

In a specific embodiment, three or four kinds of chemical reagents need to be randomly selected from the chemical reagents that meet the conditions for the test, and their surface energy parameters are put into the calculation formula of asphalt surface energy parameters for solver. When evaluating the stability of contact angle values among different testing methods, every three or four kinds of chemical reagents form a chemical reagent combination (also referred to as combination of the chemical reagents), the stability of asphalt surface energy parameters calculated by different chemical reagent combinations (also referred to as combinations of the chemical reagents) needs to be further evaluated.

In another specific embodiment, according to different testing methods, the variation coefficient of the contact angle value formed between each chemical reagent and the asphalt glass is calculated, and the testing method with minimum data discrete degree is selected. Under the different testing methods, according to the fact that asphalt is not a unipolar substance, that is, the three asphalt surface energy parameters should be greater than zero, moreover, the total energy of asphalt surface energy calculated under each chemical reagent combination shall not be greater than the total energy of chemical reagent surface energy of any chemical reagent in each chemical reagent combination. The chemical reagent combinations with obviously unreasonable data can be excluded.

In a specific embodiment, the obtaining variation coefficients of the asphalt surface energy parameters corresponding to each of the combinations of the chemical reagents and selecting a group of combinations of the chemical reagents from the combinations of the chemical reagents according to the variation coefficients of the asphalt surface energy parameters corresponding to each of the combinations of the chemical reagents, includes:

selecting the combinations of the chemical reagents corresponding to the asphalt surface energy parameters of both two kinds of asphalt being not zero from the different chemical reagents, calculating the variation coefficients of the asphalt surface energy parameters of each of the two kinds of asphalt of the respective combinations of the chemical reagents, and selecting the group of combinations of the chemical reagents according to the variation coefficients of the asphalt surface energy parameters of each of the two kinds of asphalt.

In a specific embodiment, the variation coefficient of data obtained from solver of each chemical reagent combination is calculated according to the asphalt surface energy parameters of different kinds of asphalt. In order to control the testing variables, for a specific asphalt surface energy parameter, the variation coefficient value takes the average value of the variation coefficients calculated by the different kinds of asphalt. With the help of calculation formulas of the variation coefficient C.V. evaluating data stability, the calculation formulas are:

$$C . V . = \frac{σ}{\vert μ \vert} , σ = \sqrt{\frac{\sum {( {x}_{i} - μ )}^{2}}{n}} , and μ = \frac{\sum {x}_{i}}{n} .$$

Where C.V. is the variation coefficient, σ is the standard deviation of the original data, μ is the average value of the original data, xᵢ is an observation value in the original data, and n is the number of data.

In a specific embodiment, for the asphalt surface energy parameters of the different kinds of asphalt, the variation coefficients of the data obtained from solver of the respective chemical reagent combinations are compared, and the first three chemical reagent combinations with small variation coefficients corresponding to the five asphalt surface energy parameters (γ_{S}^{Lw}, γ_{S}⁺, γ_{S}⁻, γ_{S}^{AB} and γ_{S}) are selected.

In a specific embodiment, the obtaining numbers of abnormal values of asphalt surface energy components in the group of combinations of the chemical reagents, includes:

obtaining the numbers of the abnormal values of asphalt surface energy components of respective combinations of the chemical reagents in the group of combinations of the chemical reagents according to jump degrees of asphalt surface energy components of the combinations of the chemical reagents corresponding to the asphalt surface energy parameters of both the two kinds of asphalt (70 #base asphalt and polystyrene-polybutadiene-polystyrene (SBS) modified asphalt) being not zero.

In a specific embodiment, the abnormal value is also known as an outlier, that is, some data in a group of statistical data that are obviously inconsistent with other data, the outlier can be distinguished by the jump degree testing method.

Letting X₍₁₎, X₍₂₎, . . . , X₍ₙ₋₁₎, X₍ₙ₎ be an order statistic with a sample size of n from a population distribution F (x; θ), μₖ is a point estimate value of an expectation μ that depends only on X₍₁₎, . . . , X₍ₖ₎, then

$$\frac{{μ}_{k + 1}}{{μ}_{k}}$$

is called a jump degree of μ at a point k (also referred to as the jump degree at the point k). The calculation formulas of the jump degree are:

$${μ}_{k} = \frac{{\sum }_{i = 1}^{k} {x}_{( i )} + ( n - k ) {x}_{( k )}}{k} , {μ}_{k + 1} = \frac{{\sum }_{i = 1}^{k + 1} {x}_{( i )} + [ n - ( k + 1 ) ] {x}_{( k + 1 )}}{k + 1} , {D}_{k} = \frac{{μ}_{k + 1}}{{μ}_{k}} .$$

Where μₖ and μₖ₊₁ are point estimate values of the expectation, k is the sequence of any order of statistical data, k=1, 2, 3, . . . , n, and Dₖ is the jump degree at the point k.

In a specific embodiment, the obtaining a target combination of the chemical reagents from the group of combinations of the chemical reagents according to the numbers of the abnormal values, includes: taking a combination of the chemical reagents with a least number of the abnormal values of asphalt surface energy components in the group of combinations of the chemical reagents as the target combination of the chemical reagents.

In a specific embodiment, each of the combinations of the chemical reagents includes three kinds of chemical reagents or four kinds of chemical reagents from the different chemical reagents.

It should be noted that for any group of samples composed of n number of data, after sorting all data from small to large, if there are abnormal values, they must reside at both ends of the series composed of the group of the data, and the existence of the abnormal values must make the expected point estimation produce intermittent jumps. Therefore, if there are more than one abnormal value, the maximum jump point estimated from the expected point, that is, the point with the largest jump degree, is most likely to be the starting point of the abnormal data.

There are three kinds of abnormal values in the group of data: only abnormal large value, only abnormal small value, or both abnormal small value and abnormal large value. For each case, the following steps can be used for testing the abnormal value: (1) arranging all the data in the order from small to large, and calculating the jump degree at each point; (2) finding the point with the maximum jump degree from both ends of the data; (3) if there is a significant difference between the maximum jump degree and the adjacent jump degree, the statistical data corresponding to the left side is the maximum abnormal small value, and the statistical data corresponding to the right side is the minimum abnormal large value.

With the help of jump degree analysis, the number of the abnormal values of the asphalt surface energy parameters in the group of combinations of the chemical reagents can be used to screen out the chemical reagent combination with relatively good stability of data and relatively least abnormal values. The type of the chemical reagent contained in the chemical reagent combination after three screening is the target combination of the chemical reagents, and the asphalt surface energy parameters calculated under the target combination of the chemical reagents are the target asphalt surface energy parameters.

### Embodiment 2

An embodiment of the disclosure provides a method for selecting chemical reagents in measurement of asphalt surface energy, the method includes: selecting a static drop method and a inserting plate method to measure asphalt surface energy of the two kinds of asphalt, selecting at least three kinds of chemical reagents with known surface energy parameters as the testing reagents (probe liquid) to measure contact angles between the respective asphalt slides and different chemical reagents, so as to obtain original testing data; after putting the contact angles and the surface energy parameters of the different chemical reagents into a made Excel table, when the simultaneous equations are used to calculate the asphalt surface energy parameters, the overall least square method is used to solve the contact angle calculation formula. When selecting the chemical reagent combination, the variation coefficient is used to evaluate the stability of the testing data, and the interference of abnormal values in the testing data to the stability analysis is eliminated with the help of the jump degree testing method. After many times of comparison and filtration, the chemical reagent combination with relatively good data stability and relatively least abnormal values was finally selected.

In a specific embodiment, eight kinds of chemical reagents including distilled water, formamide, ethylene glycol, glycerol, dimethyl sulfoxide, diiodomethane, benzyl alcohol and n-octanol are selected as testing reagents. The basic selection principles are as follows: first, the chemical reagent is a single homogeneous pure liquid reagent and does not dissolve or react with the asphalt material; second, the surface energy parameters of the chemical reagent are known quantities. In order to use the simultaneous equations in the contact angle calculation formula and solve it, the unknown quantities in the simultaneous equations are only three asphalt surface energy components; three, the chemical reagent can form a stable contact angle with the asphalt slide, that is, the total energy of chemical reagent surface energy is greater than the total energy of asphalt surface energy. The English letter abbreviations of the eight kinds of chemical reagents and their surface energy parameters are listed in Table 1 respectively. The different chemical reagents and their surface energy parameters are shown in Table 1.

TABLE 1

| reagent name | surface energy parameters/(erg/cm²) | | | | |
|---|---|---|---|---|---|
| (abbreviation) | γ^{LW} | γ^{AB} | γ⁺ | γ⁻ | γ_{L} |
| distilled water (W) | 21.8 | 51.0 | 25.5 | 25.5 | 72.8 |
| formamide (F) | 39.0 | 19.0 | 2.28 | 39.6 | 58.0 |
| ethylene glycol (E) | 29.0 | 19.0 | 3.0 | 30.1 | 48.0 |
| glycerol (G) | 34.0 | 30.0 | 3.92 | 57.4 | 64.0 |
| dimethyl sulfoxide (S) | 36 | 8 | 0.5 | 32 | 44 |
| diiodomethane (D) | 50.8 | 0 | 0.01 | 0 | 50.8 |
| benzyl alcohol (B) | 28.6 | 11.4 | 0.95 | 34.2 | 40 |
| n-octanol (N) | 27.5 | 0 | 0 | 3.97 | 27.5 |

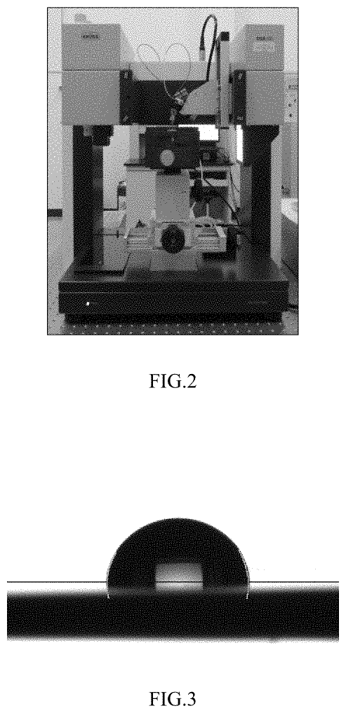

Taking the 70 #base asphalt and the SBS modified asphalt as examples, the asphalt-coated slides (also referred to as asphalt slides) were prepared, and the asphalt slides with smooth surfaces and no impurities were selected from the prepared asphalt-coated slides. After 24 hours of curing, the contact angles were measured by multiple parallel tests with the help of the static drop method and the inserting plate method. The test based on the static drop method was carried out by the optical contact angle meter (DSA100). The optical contact angle meter, as shown in FIG. 2. The basic steps of the test based on the static drop method are as follows:

S11, injection of the test reagents: turning on a system software of the static drip method after turning on all instruments in sequence and operating the instruments normally, and filling the titration system with the different testing reagents respectively;

S12, horizontal placement of the asphalt slides: the prepared asphalt slides are horizontally placed in the testing cavity, there is a visual glass window on one side of the testing cavity, the high-definition camera of the optical contact angle meter can observe the shape of the droplet of the testing reagent at any time through the visual glass window to obtain its outer contour;

S13, releasing the droplet to the surface of the asphalt slide: selecting the required chemical reagent, rotating and adjusting the needle of the droplet titration system to align with the asphalt slide in the testing cavity, and then operating the software to move the needle up and down to make the distance moderate. The release rate is generally set as V=1 microliter per minute (μL/min), and the volume of liquid dripping is set as v=0.5 microliter (μL);

S14, determination of the baseline position: a boundary line formed at the moment of contact between the droplet of the testing reagent and the asphalt slide is called the baseline. Generally, the baseline position is determined in a dynamic way, that is, when the platform is raised to the moment of contact between the surface of the asphalt slide and the droplet of the testing reagent, and the droplet will form a complete projection mirror image on the surface of the asphalt slide, and the contact line of two droplet images of the droplet and the asphalt slide is the exact position of the baseline;

S15, measurement of the contact angle: using the automatic contour capture function of the software to outline the outline of the droplet. At the same time, using the ellipse fitting method to measure the stable contact angle in a short time. The image showing the measurement of the contact angle by the static drop method is shown in FIG. 3.

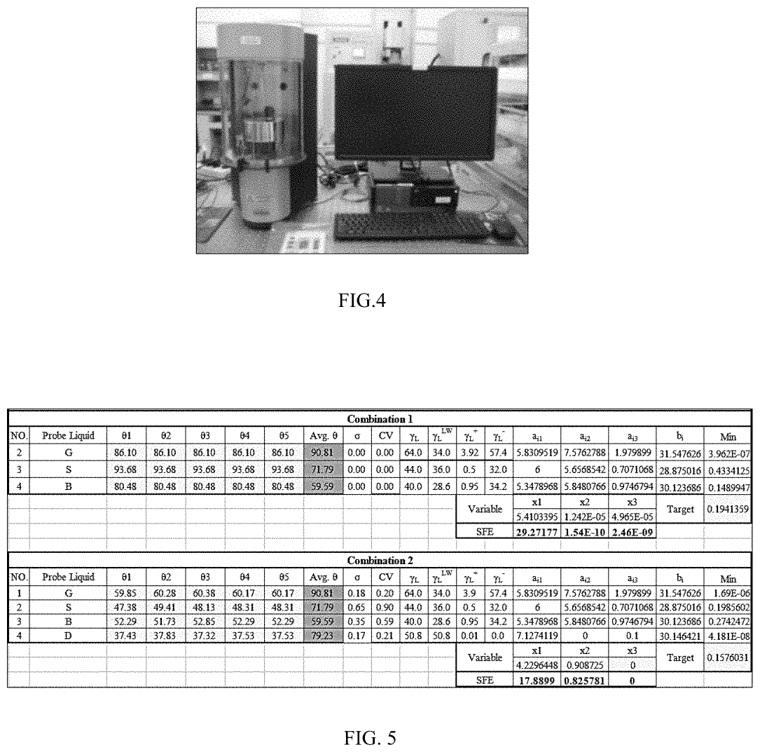

In the test based on the inserting plate method, the automatic surface tension meter (K100) is used for the test. The automatic surface tension meter as shown in FIG. 4. The basic steps of the test based on the inserting plate method are as follows:

S21, preparations before the test: about two hours before the test, opening JULABO thermostatic bath system and setting the testing temperature, and inserting the temperature probe in the instrument cavity below the liquid level of the chemical reagent;

S22, measurement of the size of the asphalt slide: taking out the cured asphalt slide from the drying oven, measuring its width and thickness with a vernier caliper, measuring each asphalt slide in parallel for 3 times and taking an average value of the measured results;

S23, horizontal fixation of the asphalt slide: fixing the asphalt slide on the sample fixture in the instrument cavity, constantly checking and adjusting to make the lower end of the asphalt slide be horizontal and the liquid level of the chemical reagent be close to the bottom end of the asphalt slide, but avoid the asphalt slide be directly immersed into the chemical reagent;

S24, measurement of the contact angle: running the K100 software, selecting the type of the chemical reagent, setting the testing depth to 2-10 millimeters (mm) and the default testing rate to 3 mm/min. after clicking to start the test, the software will automatically record and fit the contact angle value.

At the same time, in order to control the testing variables, the controllable human error and systematic error are summarized as follows: for the static drop method, the liquid volume dropped by the droplet titration system each time is set as a fixed value, and the droplet contour is fitted as quickly as possible after the droplet falls on the asphalt slide, and the contact angle value of the contour that has not been deformed by gravity is recorded, the left and right contact angles and their average values are recorded respectively. For the inserting plate method, the testing temperature of the constant temperature water bath system is set to 20° C., and each test only measures the part between 2 mm and 10 mm from the immersion of the asphalt slide into the liquid level of the chemical reagent, and the bottom end of the asphalt slide shall be parallel to the liquid level of the chemical reagent as far as possible. Each kind of the asphalt slides is prepared from the asphalt of the same batch and place of origin, and the curing time in the drying oven is the same. For the same testing method, the same kind of the asphalt slide and the same kind of the chemical reagent, measurements of three parallel test are carried out, and the final contact angle value is the average of the three measured results. The measured contact angle values (also referred to as final contact angle values) are recorded in Table 2 and Table 3. The contact angle values obtained by the static drop method and the inserting plate method are shown in Table 2 and Table 3 respectively.

TABLE 2

| | | testing method | | | | | | | |
|---|---|---|---|---|---|---|---|---|---|
| | | static drop method | | | | | | | |
| | | reagent type | | | | | | | |
| | | W | F | E | G | S | D | B | N |
| contact | 70# base | 99.62 | 84.92 | 77.78 | 104.12 | 68.20 | 45.43 | 56.68 | 11.71 |
| angle/(°) | asphalt | | | | | | | | |
| | SBS | 98.27 | 80.48 | 76.06 | 100.52 | 64.57 | 47.08 | 52.99 | 9.24 |
| | modified | | | | | | | | |
| | asphalt | | | | | | | | |

TABLE 3

| | | testing method | | | | | | | |
|---|---|---|---|---|---|---|---|---|---|
| | | inserting plate method | | | | | | | |
| | | reagent type | | | | | | | |
| | | W | F | E | G | S | D | B | N |
| contact | 70# base | 104.33 | 91.96 | 87.64 | 95.20 | 73.44 | 78.21 | 66.54 | 22.84 |
| angle/(°) | asphalt | | | | | | | | |
| | SBS | 101.43 | 89.06 | 91.30 | 90.81 | 71.79 | 79.23 | 59.59 | 12.82 |
| | modified | | | | | | | | |
| | asphalt | | | | | | | | |

For the contact angle measured by the test based on the inserting plate method, because a layer of reagent liquid film has been attached to the surface of asphalt film during the withdrawal of the asphalt slide from the testing reagent, the difference between backward angle and forward angle is obvious due to the influence of liquid self-weight and surface tension, so only the value of forward angle is recorded in the table.

The image showing Excel calculation table is shown in FIG. 5. According to the different types of randomly selected chemical reagents, the table is divided into upper and lower parts. The upper part is to randomly select three kinds of chemical reagents from eight kinds of chemical reagents and put them into equations for simultaneous solution, with a total type of the chemical reagent combinations is C₈³=56. In the lower part, four kinds of the chemical reagents are randomly selected from eight kinds of chemical reagents, with a total type of the chemical reagent combinations is C₈⁴=70. In the both parts, the formula that can form the calculation formula of stable contact angle is taken as the calculation formula.

In a specific embodiment, a column marked Probe Liquid are the English abbreviations of the chemical reagents, aᵢ₁, aᵢ₂ and aᵢ₃ are \sqrt{γ_{L}^{LW}}, \sqrt{γ_{L}⁻}, and \sqrt{γ_{L}⁺} respectively, bᵢ is

$$\frac{( 1 + cos θ ) {γ}_{L}}{2} ,$$

Min represents a fitting error, Target represents an average value of the sum of three fitting errors, x1, x2 and x3 are \sqrt{γ_{S}^{LW}}, \sqrt{γ_{S}⁺}, and \sqrt{γ_{S}⁻} respectively. SFE represents each calculated value corresponding to each asphalt surface energy parameter. In the contact angle calculation formula, the surface energy parameters of different chemical reagents and the corresponding contact angle values are input respectively, and the asphalt surface energy parameters can be obtained by simultaneous equations and linear solver.

In another specific embodiment, the function of Solver in Excel software is used to calculate the asphalt surface energy parameters and sort out and record the data. The basic steps are as follows: the three asphalt surface energy parameters x1, x2 and x3 to be solved are set as variable cells, and the average value of the sum of the three Min values is set as the target value; before each solver, it only needs to change the type of testing reagent, three surface energy parameters of the testing reagent and the contact angle value.

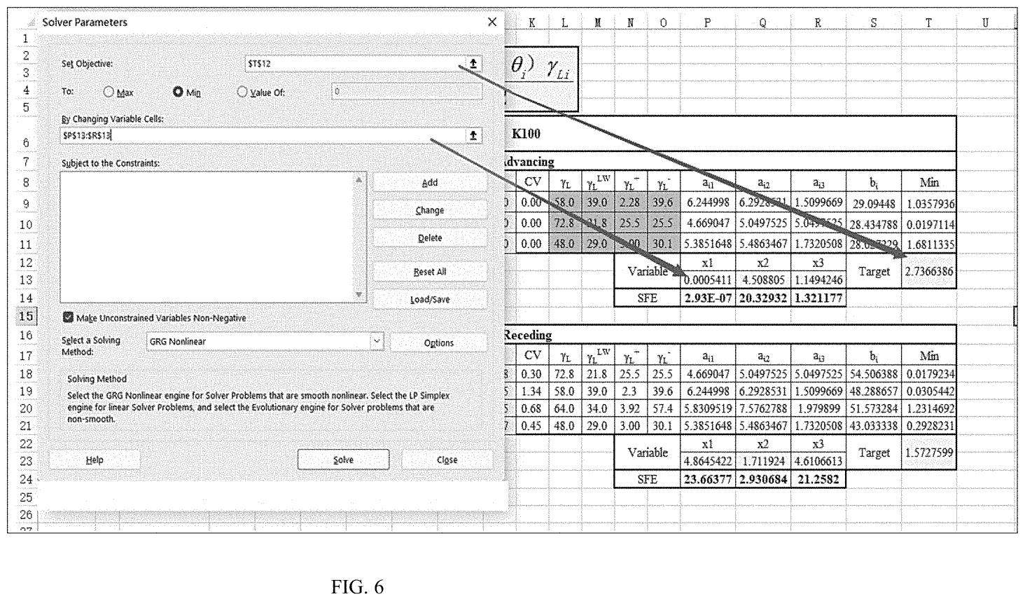

Clicking the “File” menu in an upper right corner of the toolbar in the Excel table, and continuing to click “Options”—“Add ins”—“Go”. After the window of “Add-ins available” is popped up, checking the “Solver Add-in”. At this time, the “Solver” option will appear under the “Data” page of the toolbar; opening the Solver Parameters, of Solver, filling in “Set Objective” and “By Changing Variable Cells”, checking “Make Unconstrained Variables Non-Negative”, and the solution method is “GRG Nonlinear”, and then you can solve it. The operation interface of Solver in Excel is shown in FIG. 6.

Repeating the basic steps of the solver, and recording the calculated asphalt surface energy parameters in Tables 4 to 7 according to different testing methods and asphalt types. The asphalt surface energy parameters are calculated according to the different testing methods and the asphalt types, as shown in Tables 4-7.

TABLE 4

| | testing | | | | | |
|---|---|---|---|---|---|---|
| | method of | | | | | |
| | contact | | | | | |
| asphalt type | angle | asphalt surface energy | | | | |
| 70# base | static drop | parameters/(erg/cm²) | | | | |
| asphalt | method | γ_{S}^{LW} | γ_{S}⁺ | γ_{S}⁻ | γ_{S}^{AB} | γ_{S} |
| chemical | WFE | 24.05 | 0.13 | 4.27 | 1.48 | 25.53 |
| reagent | WFB | 33.57 | 3.39 | 0.46 | 2.49 | 36.06 |
| combination | WEB | 33.57 | 0.80 | 1.03 | 1.81 | 35.38 |
| | WSB | 33.57 | 1.14 | 0.68 | 1.75 | 35.33 |
| | WBN | 23.64 | 0.81 | 0.60 | 1.39 | 25.02 |
| | FES | 24.56 | 0.14 | 1.39 | 0.89 | 25.44 |
| | WFEG | 21.88 | 1.46 | 2.58 | 3.89 | 25.77 |
| | WFES | 29.04 | 0.27 | 2.10 | 1.51 | 30.55 |

TABLE 5

| | testing | | | | | |
|---|---|---|---|---|---|---|
| | method of | | | | | |
| | contact | | | | | |
| asphalt type | angle | asphalt surface energy | | | | |
| 70# base | inserting | parameters/(erg/cm²) | | | | |
| asphalt | plate method | γ_{S}^{LW} | γ_{S}⁺ | γ_{S}⁻ | γ_{S}^{AB} | γ_{S} |
| chemical | WFE | 21.53 | 0.24 | 2.57 | 1.57 | 23.11 |
| reagent | WFG | 24.91 | 0.15 | 1.55 | 0.96 | 25.86 |
| combination | WFS | 22.20 | 0.05 | 2.82 | 0.76 | 22.96 |
| | WFD | 20.11 | 0.43 | 3.23 | 2.37 | 22.48 |
| | WFB | 27.34 | 3.30 | 0.98 | 3.59 | 30.93 |
| | WES | 22.20 | 0.60 | 1.62 | 1.98 | 24.18 |
| | WGS | 24.91 | 0.48 | 2.37 | 2.14 | 27.05 |
| | WGD | 24.91 | 0.15 | 1.42 | 0.91 | 25.82 |
| | WGB | 27.34 | 3.38 | 0.74 | 3.15 | 30.49 |
| | WSD | 22.20 | 0.10 | 2.47 | 1.00 | 23.20 |
| | WSB | 27.34 | 0.77 | 0.56 | 1.31 | 28.65 |
| | WDB | 27.34 | 0.56 | 1.60 | 1.89 | 29.22 |
| | WBN | 27.34 | 0.01 | 0.50 | 0.12 | 27.46 |
| | FEG | 21.53 | 0.12 | 1.72 | 0.90 | 22.43 |
| | FGS | 22.20 | 1.17 | 0.18 | 0.92 | 23.12 |
| | GSD | 22.20 | 0.14 | 1.43 | 0.89 | 23.09 |
| | WFEG | 21.53 | 0.24 | 2.57 | 1.57 | 23.11 |
| | WFES | 21.53 | 0.24 | 2.57 | 1.57 | 23.11 |
| | WFGS | 19.22 | 0.02 | 1.50 | 0.36 | 19.58 |
| | WFGD | 18.27 | 0.04 | 1.62 | 0.51 | 18.78 |
| | WFGB | 7.98 | 1.64 | 2.35 | 3.92 | 11.90 |
| | WFGN | 7.98 | 1.64 | 2.35 | 3.92 | 11.90 |
| | WFSD | 18.28 | 0.10 | 1.33 | 0.73 | 19.01 |
| | WFSB | 18.28 | 0.10 | 1.33 | 0.73 | 19.01 |
| | WFSN | 18.28 | 0.10 | 1.33 | 0.73 | 19.02 |
| | WFDB | 18.28 | 0.10 | 1.33 | 0.73 | 19.01 |
| | WFDN | 18.28 | 0.10 | 1.33 | 0.73 | 19.01 |
| | WFBN | 25.06 | 0.01 | 0.50 | 0.12 | 25.18 |
| | WEGS | 18.27 | 0.05 | 1.55 | 0.56 | 18.83 |
| | WEGD | 18.27 | 0.05 | 1.55 | 0.56 | 18.83 |
| | WEBN | 25.06 | 0.01 | 0.50 | 0.12 | 25.18 |
| | WGSD | 18.27 | 0.05 | 1.55 | 0.56 | 18.83 |
| | WGSB | 18.86 | 0.06 | 1.35 | 0.57 | 19.43 |
| | WGSN | 18.86 | 0.06 | 1.35 | 0.57 | 19.43 |
| | WGDB | 18.33 | 0.55 | 0.52 | 1.07 | 19.41 |
| | WGDN | 16.81 | 1.37 | 0.5 | 1.66 | 18.47 |

TABLE 6

| | testing | | | | | |
|---|---|---|---|---|---|---|
| | method of | | | | | |
| | contact | | | | | |
| asphalt type | angle | asphalt surface energy | | | | |
| SBS modified | static drop | parameters/(erg/cm²) | | | | |
| asphalt | method | γ_{S}^{LW} | γ_{S}⁺ | γ_{S}⁻ | γ_{S}^{AB} | γ_{S} |
| chemical | WFE | 30.58 | 1.05 | 2.96 | 3.53 | 34.11 |
| reagent | WEG | 30.58 | 1.05 | 2.34 | 3.14 | 33.71 |
| combination | WES | 30.58 | 1.26 | 2.09 | 3.25 | 33.83 |
| | WGS | 27.47 | 0.31 | 3.25 | 2.00 | 29.46 |
| | FGS | 27.47 | 0.29 | 2.95 | 1.86 | 29.33 |
| | SDB | 35.49 | 0.27 | 3.28 | 1.90 | 37.39 |
| | WFEG | 25.48 | 0.16 | 2.26 | 0.60 | 26.08 |
| | WFGB | 25.40 | 0.24 | 2.23 | 0.73 | 26.13 |
| | WFGN | 25.38 | 0.26 | 2.26 | 1.15 | 26.53 |

TABLE 7

| | testing | | | | | |
|---|---|---|---|---|---|---|
| | method of | | | | | |
| | contact | | | | | |
| asphalt type | angle | asphalt surface energy | | | | |
| SBS modified | inserting | parameters/(erg/cm²) | | | | |
| asphalt | plate method | γ_{S}^{LW} | γ_{S}⁺ | γ_{S}⁻ | γ_{S}^{AB} | γ_{S} |
| chemical | WFE | 22.28 | 0.74 | 4.06 | 3.46 | 25.74 |
| reagent | WFG | 29.27 | 0.27 | 2.97 | 1.81 | 31.08 |
| combination | WFS | 23.16 | 0.07 | 2.57 | 0.86 | 24.02 |
| | WFD | 22.28 | 0.58 | 2.30 | 2.30 | 24.58 |
| | WEG | 29.27 | 0.03 | 5.58 | 0.82 | 30.09 |
| | WES | 23.16 | 0.09 | 2.91 | 1.02 | 24.18 |
| | WGS | 29.27 | 0.68 | 1.89 | 2.26 | 31.53 |
| | WGD | 29.27 | 0.34 | 1.70 | 1.52 | 30.79 |
| | WSD | 23.16 | 0.07 | 2.57 | 0.86 | 24.02 |
| | FEG | 22.28 | 0.74 | 4.06 | 3.46 | 25.74 |
| | FES | 22.28 | 0.74 | 4.06 | 3.46 | 25.74 |
| | FEB | 22.28 | 0.74 | 4.06 | 3.46 | 25.74 |
| | FEN | 22.28 | 0.74 | 4.06 | 3.46 | 25.74 |
| | FGS | 23.16 | 3.59 | 0.74 | 3.26 | 26.42 |
| | FGD | 22.28 | 0.74 | 4.06 | 3.46 | 25.74 |
| | FSD | 22.28 | 0.74 | 4.06 | 3.46 | 25.74 |
| | FDB | 22.28 | 0.74 | 4.06 | 3.46 | 25.74 |
| | FDN | 22.28 | 0.74 | 4.06 | 3.46 | 25.74 |
| | EGS | 23.16 | 3.59 | 0.74 | 3.26 | 26.42 |
| | GSD | 23.16 | 0.15 | 4.28 | 1.62 | 24.78 |
| | WFEG | 5.99 | 3.40 | 2.80 | 6.17 | 12.16 |
| | WFES | 4.70 | 4.59 | 2.67 | 7.00 | 11.70 |
| | WFGS | 5.40 | 6.24 | 1.28 | 5.66 | 11.06 |
| | WFGD | 17.72 | 0.34 | 1.71 | 1.52 | 19.24 |
| | WFGB | 6.30 | 3.23 | 2.76 | 5.97 | 12.27 |
| | WFSD | 17.72 | 0.21 | 2.03 | 1.32 | 19.04 |
| | WFDB | 17.86 | 2.56 | 0.05 | 0.71 | 18.57 |
| | WFDN | 17.85 | 2.38 | 0.11 | 1.01 | 18.86 |
| | WFBN | 24.23 | 0.42 | 0.34 | 0.75 | 24.98 |
| | WEGS | 15.40 | 0.59 | 1.91 | 2.13 | 17.53 |
| | WESB | 5.21 | 6.44 | 1.35 | 5.90 | 11.11 |
| | WGSD | 17.73 | 0.21 | 2.05 | 1.31 | 19.05 |
| | WGSB | 15.13 | 0.66 | 1.89 | 2.22 | 17.35 |
| | WGDB | 17.81 | 1.38 | 0.49 | 1.65 | 19.46 |
| | WGDN | 18.86 | 2.44 | 0.08 | 0.86 | 19.72 |
| | WGBN | 25.19 | 0.17 | 0.52 | 0.60 | 25.79 |
| | WSDB | 17.81 | 1.37 | 0.50 | 1.66 | 19.46 |
| | WSDN | 17.72 | 0.21 | 2.09 | 1.33 | 19.05 |
| | WSBN | 25.04 | 0.21 | 0.48 | 0.64 | 25.68 |
| | WDBN | 17.86 | 2.57 | 0.07 | 0.85 | 18.70 |
| | FEGB | 5.60 | 5.09 | 0.11 | 1.50 | 7.10 |
| | FGSB | 10.80 | 2.58 | 0.01 | 0.36 | 11.16 |
| | EGSB | 13.34 | 1.43 | 0.09 | 0.71 | 14.05 |

In order to facilitate the first filtration of the chemical reagent combinations, recording and sorting out the data after considering the following conditions: WFE represents the chemical reagent combination of “distilled water+formamide+ethylene glycol”, WFEG represents the chemical reagent combination of “distilled water+formamide+ethylene glycol+glycerol”, and so on. Since asphalt is not a unipolar substance in practice, the three asphalt surface energy parameters should be greater than zero, and some calculated values of the asphalt surface energy parameters are zero. Therefore, in order to facilitate the analysis of data stability and abnormal values in the next description and eliminate the interference of obviously unreasonable data, only the chemical reagent combination with calculated values of the asphalt surface energy parameters that are not zero will be considered below. The calculated total energy of asphalt surface energy under each chemical reagent combination shall not be greater than the total energy of chemical reagent surface energy of any chemical reagent in the chemical reagent combination, otherwise it is excluded.

In a specific embodiment, the variation coefficient is used to evaluate the stability of the data obtained from testing methods, the first filtration of chemical reagent combinations is completed, then the data stability of each chemical reagent combinations is evaluated, and the second filtration is completed.

According to different testing methods, the variation coefficient of the contact angle formed between each chemical reagent and the asphalt slide is calculated, and the testing method with minimum data discrete degree is selected. The variation coefficient values of each group of data are listed in Table 8 and Table 9. Table 8 and Table 9 correspond to the variation coefficients of contact angle values obtained by the static drop method and the inserting plate method respectively.

TABLE 8

| | testing method | | | | | | | | | | | | | | | |
|---|---|---|---|---|---|---|---|---|---|---|---|---|---|---|---|---|
| | static drop method | | | | | | | | | | | | | | | |
| | asphalt type | | | | | | | | | | | | | | | |
| | 70# base asphalt | | | | | | | | SBS modified asphalt | | | | | | | |
| | reagent type | | | | | | | | | | | | | | | |
| | W | F | E | G | S | D | B | N | W | F | E | G | S | D | B | N |
| variation | 0.85 | 0.61 | 1.86 | 1.65 | 2.41 | 1.79 | 3.55 | 10.12 | 0.34 | 0.28 | 0.96 | 0.60 | 2.09 | 2.62 | 1.68 | 5.43 |
| coefficient | | | | | | | | | | | | | | | | |
| (%) | | | | | | | | | | | | | | | | |

TABLE 9

| | testing method | | | | | | | | | | | | | | | |
|---|---|---|---|---|---|---|---|---|---|---|---|---|---|---|---|---|
| | inserting plate method | | | | | | | | | | | | | | | |
| | asphalt type | | | | | | | | | | | | | | | |
| | 70# base asphalt | | | | | | | | SBS modified asphalt | | | | | | | |
| | reagent type | | | | | | | | | | | | | | | |
| | W | F | E | G | S | D | B | N | W | F | E | G | S | D | B | N |
| variation | 0.97 | 0.22 | 0.37 | 1.51 | 2.05 | 0.59 | 2.44 | 3.95 | 0.29 | 0.56 | 0.82 | 0.53 | 0.48 | 0.54 | 2.20 | 2.85 |
| coefficient | | | | | | | | | | | | | | | | |
| (%) | | | | | | | | | | | | | | | | |

Taking the contact angle value between the distilled water and 70 #base asphalt measured in the test based on the static drop method as an example, the variation coefficient is calculated as:

$$C . V . = \frac{σ}{\vert μ \vert} = \frac{\sqrt{\frac{1}{3} [ {( 100.55 - 99.62 )}^{2} + {( 99.4 - 99.62 )}^{2} + {( 98.9 - 99.62 )}^{2} ]}}{\vert \frac{1}{3} \times ( 100.55 + 99.4 + 98.90 ) \vert} = 0.85 \% .$$

By comparing the C.V. values of the two methods, except for three cases, the variation coefficients of the contact angle values measured by the static drop method are greater than that measured by the inserting plate method, that is, the discrete degree of the contact angle values measured by the static drop method is greater than that measured by the inserting plate method. The three cases include 70 #base asphalt+W, SBS modified asphalt+F and SBS modified asphalt+B.

During the specific implementation, there are many human interference factors in the test based on the static drop method, resulting in data frequently produce large errors. From Table 4 to Table 7, it can be seen that the contact angle values measured by the static drop method are not ideal, so the asphalt surface energy parameters calculated in the solver are often zero, and too many chemical reagent combinations have to be excluded. Compared with the static drop method, the contact angle values measured by the inserting plate method are stable, the variation coefficients of the contact angle values obtained from multiple parallel tests is less than 4%, and there are few human interference factors in the process of testing operation, which requires high data accuracy and the level of making asphalt slides. Therefore, in order to make the conclusion more universal and effective, only the data stability of the chemical reagent combinations filtered under the test based on the inserting board method are analyzed and evaluated.

It can be seen from Tables 5 and 7 that there are 24 kinds of chemical reagent combinations corresponding to the asphalt surface energy parameters of two kinds of asphalt being not zero at the same time. According to the asphalt surface energy parameters of the different kinds of asphalt, the variation coefficients of the obtained data (contact angle) obtained from solver of chemical reagent combinations are calculated respectively and recording them in Table 10. For a specific asphalt surface energy parameter, the value of the variation coefficient is the average value of the variation coefficients calculated by the two kinds of asphalts. It should be noted that since the test is repeated for three times, the data obtained from the solver of each chemical reagent combination is three values, and the calculating variation coefficient is calculating the variation coefficients of the three values respectively. The variation coefficient of contact angle corresponding to each chemical reagent combination is shown in Table 10.

TABLE 10

asphalt surface energy parameters

| | | | | | |
|---|---|---|---|---|---|
| reagent | γ_{S}^{LW} | γ_{S}⁺ | γ_{S}⁻ | γ_{S}^{AB} | γ_{S} |

| | |
|---|---|
| combination | variation coefficient (%) |

| | | | | | |
|---|---|---|---|---|---|
| WFE | 2.42 | 72.15 | 31.78 | 53.14 | 7.61 |
| WFG | 11.38 | 40.41 | 44.43 | 43.40 | 12.96 |
| WFS | 2.99 | 23.57 | 70.59 | 41.13 | 4.99 |
| WFD | 7.24 | 21.00 | 23.78 | 2.12 | 6.31 |
| WGS | 11.38 | 24.38 | 15.93 | 3.86 | 10.82 |
| WGD | 11.38 | 54.84 | 12.69 | 35.50 | 12.42 |
| WSD | 2.99 | 24.96 | 2.81 | 10.64 | 2.46 |
| FEG | 2.42 | 101.95 | 57.25 | 83.04 | 9.72 |
| FGS | 2.99 | 71.90 | 86.08 | 79.17 | 9.42 |
| GSD | 2.99 | 4.88 | 6.56 | 8.73 | 3.19 |
| WFEG | 79.86 | 122.77 | 6.06 | 84.05 | 43.91 |
| WFES | 90.74 | 127.37 | 2.70 | 89.61 | 46.36 |
| WFGS | 79.38 | 140.52 | 11.19 | 124.51 | 39.32 |
| WFGD | 2.16 | 111.65 | 3.82 | 70.36 | 1.71 |
| WFGB | 16.64 | 46.17 | 11.35 | 29.31 | 2.16 |
| WFSD | 2.20 | 50.18 | 29.46 | 40.70 | 0.11 |
| WFDB | 1.64 | 130.79 | 131.17 | 1.96 | 1.66 |
| WFDN | 1.68 | 130.02 | 119.82 | 22.76 | 0.56 |
| WFBN | 2.38 | 134.84 | 26.94 | 102.41 | 0.56 |
| WEGS | 12.05 | 119.32 | 14.71 | 82.54 | 5.06 |
| WGSD | 2.12 | 87.03 | 19.64 | 56.72 | 0.82 |
| WGSB | 15.52 | 117.85 | 23.57 | 83.64 | 8.00 |
| WGDB | 2.03 | 60.82 | 4.20 | 30.16 | 0.18 |
| WGDN | 8.13 | 39.72 | 102.41 | 44.90 | 4.67 |

It can be seen from Table 10 that for the asphalt surface energy parameters of different kinds of asphalt, the variation coefficients of the data obtained from the solver of the respective chemical reagent combinations are compared as follows (only the first ten with smaller values are taken).

γ_{S}^{LW}: WFDB<WFDN<WGDB<WGSD<WFGD<WFSD<WFBN<WFE=FEG<WSD=WFS=FGS=GSD;

γ_{S}⁺: GSD<WFD<WFS<WGS<WSD<WGDN<WFG<WFGB<WFSD<WGD;

γ_{S}⁻: WFES<WSD<WFGD<WGDB<WFEG<GSD<WFGS<WFGB<WGD<WEGS;

γ_{S}^{AB}: WFDB<WFD<WGS<GSD<WSD<WFDN<WFGB<WGDB<WGD<WFSD;

γ_{S}: WFSD<WGDB<WFBN=WFDN<WGSD<WFDB<WFGD<WFGB<WSD<GSD.

The comparison shows that there is no unique chemical reagent combination with the smallest variation coefficient among the five kinds of asphalt surface energy components. Therefore, the first three chemical reagent combinations with the smaller variation coefficients corresponding to the respective surface energy components are selected as WFD, WFS, WSD, GSD, WGS, WEFS, WFGD, WFSD, WFDB, WFDN, WFBN and WGDB.

Because there are more or less abnormal values in the data, it is necessary to test the abnormal values of the above twelve chemical reagent combinations, so as to eliminate the interference of abnormal values on the data stability analysis, ensure the accuracy and reliability of the variation coefficients in measuring the discrete degree of each group of data, and finally select the chemical reagent combination with good data stability and less abnormal values. Finally, taking the nonpolar component γ_{S}^{LW} of the 70 #base asphalt as an example, the jump degree is used to analyze whether there are abnormal values in the data of the above twelve reagent combinations.

According to the testing steps of the jump degree, the calculated values obtained from solver of the nonpolar component γ_{S}^{LW} of 70 #base asphalt by all chemical reagent combinations in Table 10 are arranged from small to large as follows: 7.98, 16.81, 18.27, 18.27, 18.28, 18.28, 18.28, 18.33, 18.86, 19.22, 20.11, 21.53, 21.53, 21.53, 22.2, 22.2, 22.2, 24.91, 24.91, 24.91, 25.06, followed by, from the calculation formula of the jump degree:

$${μ}_{1} = 191.52 , {μ}_{2} = 197.31 , {μ}_{3} = 142.24 , … , {μ}_{23} = 21.11 , {μ}_{24} = 20.24 ,$$

$$\frac{{μ}_{2}}{{μ}_{1}} = 1.03 , \frac{{μ}_{3}}{{μ}_{2}} = 0.72 , \frac{{μ}_{4}}{{μ}_{3}} = 0.75 , … , \frac{{μ}_{23}}{{μ}_{22}} = 0.80 , \frac{{μ}_{24}}{{μ}_{23}} = 0.83 .$$

It can be seen from the above calculation that, except for

$$\frac{{μ}_{2}}{{μ}_{1}} ,$$

all other jump degrees are less than 1, so the first data 7.98 is an abnormal small value, that is, there is an abnormal value in the calculated value of the non-polar component γ_{S}^{LW} of the 70 #base asphalt by the chemical reagent combination WFGB. Similarly, the testing method of the jump degree is used to analyze the twelve kinds of chemical reagent combinations selected through the variation coefficients and judge the numbers of abnormal values in the data obtained by the respective chemical reagent combinations, as shown in Table 11.

TABLE 11

| | jump degree testing result | | | | | | | | | | | |
|---|---|---|---|---|---|---|---|---|---|---|---|---|
| | chemical reagent combination | | | | | | | | | | | |
| | WFD | WFS | WSD | WGS | GSD | WFES | WFGD | WFSD | WFDB | WFDN | WFBN | WGDB |
| number of | 1 | 3 | 2 | 1 | 0 | 5 | 1 | 1 | 2 | 2 | 5 | 3 |
| abnormal values | | | | | | | | | | | | |

It can be seen from Table 11 that the number of the abnormal values in the data calculated by the chemical reagent combination GSD is minimum and zero, that is, there is no abnormal value. Combined with the analysis of the variation coefficient of the data calculated by each chemical reagent combination, the data stability of the chemical reagent combination GSD is relatively good and the abnormal value is relatively minimum.

Finally, glycerol, dimethyl sulfoxide and diiodomethane are selected, and the calculated values of the asphalt surface energy parameters are recorded in Table 12. The calculated values of the asphalt surface energy parameters are shown in Table 12.

TABLE 12

| | equation | reagent | | | | | | |
|---|---|---|---|---|---|---|---|---|
| testing | solving | combination | asphalt | asphalt surface energy parameters | | | | |
| method | method | type | type | γ_{S}^{LW} | γ_{S}⁺ | γ_{S}⁻ | γ_{S}^{AB} | γ_{S} |
| inserting | overall | GSD | 70# base | 22.2 | 0.14 | 1.43 | 0.89 | 23.09 |
| plate | least | | asphalt | | | | | |
| method | square | | SBS | 23.16 | 0.15 | 4.28 | 1.62 | 24.78 |
| | method | | modified | | | | | |
| | | | asphalt | | | | | |

### Embodiment 3

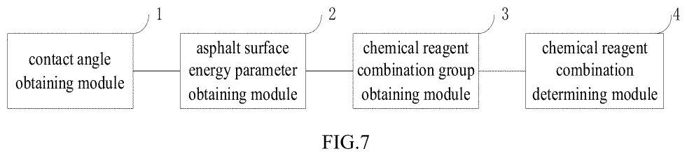

An embodiment of the disclosure provided a system for selecting chemical reagents in measurement of asphalt surface energy, as shown in FIG. 7, the system includes: a contact angle obtaining module 1, an asphalt surface energy parameter obtaining module 2, a chemical reagent combination group obtaining module 3 and a chemical reagent combination determining module 4.

The contact angle obtaining module 1 is configured to select different chemical reagents and obtain contact angle values formed between the respective chemical reagents and asphalt slides.

The asphalt surface energy parameter obtaining module 2 is configured to obtain asphalt surface energy parameters corresponding to each of combinations of the chemical reagents according to the contact angle values formed between the respective chemical reagents and the asphalt slides.

The chemical reagent combination group obtaining module 3 is configured to obtain variation coefficients of the asphalt surface energy parameters corresponding to each of the combinations of the chemical reagents and select a group of combinations of the chemical reagents from the combinations of the chemical reagents according to the variation coefficients of the asphalt surface energy parameters corresponding to each of the combinations of the chemical reagents.

The chemical reagent combination determining module 4 is configured to obtain numbers of abnormal values of asphalt surface energy components in the group of combinations of the chemical reagents and obtain a target combination of the chemical reagents according to the numbers of the abnormal values of asphalt surface energy components.

The contact angle obtaining module 1, the asphalt surface energy parameter obtaining module 2, the chemical reagent combination group obtaining module 3 and the chemical reagent combination determining module 4 are software modules stored in one or more memories and executable by one or more processors coupled to the one or more memories.

The method and system for selecting chemical reagents in measurement of asphalt surface energy, by selecting the different chemical reagents and obtaining the contact angle values formed between the respective chemical reagents and the asphalt slides, obtaining the asphalt surface energy parameters corresponding to each of the combinations of the chemical reagents according to the contact angle values, obtaining the variation coefficients of the asphalt surface energy parameters corresponding to each of the combinations of the chemical reagents and selecting the group of combinations of the chemical reagents according to the variation coefficients, and obtaining the numbers of the abnormal values of asphalt surface energy components in the group of combinations of the chemical reagents and obtaining the target combination of the chemical reagents according to the numbers of the abnormal values of asphalt surface energy components, the combination of the chemical reagents with high stability of testing data can be selected.

The technical scheme of the disclosure uses the overall least square method to solve the equations, which can reduce the error between the calculated value and the actual value of each asphalt surface energy parameter, and more conform to the geometric significance represented by the equations composed of three basic unknown equations in three-dimensional space. Therefore, the three asphalt surface energy parameters obtained by solving the equations are more reasonable and closer to the actual value, which provides a more accurate data basis for data stability evaluation.

In the aspect of analyzing the stability of data and then judging whether the data is reasonable and effective, a new analysis method is introduced to evaluate the stability of data in the measurement of asphalt surface energy, that is, the variation coefficient of, a commonly used digital feature in statistics, is used to analyze the fluctuation size of each group of data, and the interference of abnormal values in each group of data to stability analysis is eliminated by means of the jump degree testing method. Its significance is that the variation coefficient and the jump degree are applied to the testing data analysis of pavement asphalt surface energy parameters for the first time. The purpose is to filter the data of each group with great differences, so as to provide a basis for the testing design of accurate calculation of surface energy parameters.

Through the technical scheme of the disclosure, the chemical reagent combination with stable testing data is selected, which provides a reasonable and effective method basis for selecting chemical reagents for testers engaged in measuring asphalt surface energy parameters, and can be better applied to the performance test in the direction of pavement asphalt.

The above are only the specific embodiments of the disclosure, but the protection scope of the disclosure is not limited to this. Any change or replacement that can be easily thought of by the person skilled in the related art within the technical scope disclosed by the disclosure should be covered by the protection scope of the disclosure.

What is claimed is:

1. A method for selecting chemical reagents in measurement of asphalt surface energy, comprising:
selecting different chemical reagents and obtaining contact angle values formed between the respective chemical reagents and asphalt slides;
obtaining asphalt surface energy parameters corresponding to each of combinations of the chemical reagents according to the contact angle values formed between the respective chemical reagents and the asphalt slides;
obtaining variation coefficients of the asphalt surface energy parameters corresponding to each of the combinations of the chemical reagents and selecting a group of combinations of the chemical reagents from the combinations of the chemical reagents according to the variation coefficients of the asphalt surface energy parameters corresponding to each of the combinations of the chemical reagents; and
obtaining numbers of abnormal values of asphalt surface energy components in the group of combinations of the chemical reagents and obtaining a target combination of the chemical reagents from the group of combinations of the chemical reagents according to the numbers of the abnormal values of the asphalt surface energy components.

2. The method according to claim 1, wherein the obtaining contact angle values formed between the respective chemical reagents and asphalt slides, comprises:
calculating variation coefficients of the respective contact angle values formed between the respective chemical reagents and asphalt slides under different testing methods to obtain one testing method with minimum data discrete degree from the different testing methods, and obtaining the contact angle values formed between the respective chemical reagents and the asphalt slides under the one testing method with minimum data discrete degree.

3. The method according to claim 1, wherein the obtaining asphalt surface energy parameters corresponding to each of combinations of the chemical reagents according to the contact angle values formed between the respective chemical reagents and the asphalt slides, comprises:
obtaining multiple values of the asphalt surface energy parameters corresponding to each of the combinations of the chemical reagents according to the contact angle values formed between the respective chemical reagents and the asphalt slides, and obtaining the asphalt surface energy parameters corresponding to each of the combinations of the chemical reagents according to the multiple values of the asphalt surface energy parameters and by concurrently taking a minimum fitting error of the asphalt surface energy parameters as a target value.

4. The method according to claim 3, wherein the obtaining multiple values of the asphalt surface energy parameters corresponding to each of the combinations of the chemical reagents according to the contact angle values formed between the respective chemical reagents and the asphalt slides, comprises:
obtaining the multiple values of the asphalt surface energy parameters corresponding to each of the combinations of the chemical reagents according to the contact angle values formed between the respective chemical reagents and the asphalt slides and a calculation formula of asphalt surface energy parameters, wherein the calculation formula of asphalt surface energy parameters is:
$$\sqrt{{γ}_{S}^{-} {γ}_{L}^{+}} + \sqrt{{γ}_{S}^{+} {γ}_{L}^{-}} + \sqrt{{γ}_{S}^{L w} {γ}_{L}^{L w}} = \frac{( 1 + cos θ ) {γ}_{L}}{2} ,$$
where γ_{S}^{Lw} is a nonpolar component of asphalt surface energy, γ_{L}^{Lw} is a nonpolar component of chemical reagent surface energy, γ_{S}⁻ is a polar alkali component of asphalt surface energy, γ_{S}⁺ is a polar acid component of asphalt surface energy, γ_{L}⁻ is a polar alkali component of chemical reagent surface energy, γ_{L}⁺ is a polar acid component of chemical reagent surface energy, γ_{L} is a total energy of chemical reagent surface energy, and θ is a contact angle.

5. The method according to claim 4, wherein the obtaining the asphalt surface energy parameters corresponding to each of the combinations of the chemical reagents according to the multiple values of the asphalt surface energy parameters and by concurrently taking a minimum fitting error of the asphalt surface energy parameters as a target value, comprises:
obtaining the asphalt surface energy parameters corresponding to each of the chemical reagent combinations according to multiple values of each of the asphalt surface energy parameters and by concurrently taking the minimum fitting error as the target value, wherein the minimum fitting error is calculated as that:
$$Min = \frac{\vert ( \sqrt{{γ}_{S}^{L W} {γ}_{L}^{L W}} + \sqrt{{γ}_{S}^{+} {γ}_{L}^{-}} + \sqrt{{γ}_{S}^{-} {γ}_{L}^{+}} ) - \frac{{γ}_{L} ( 1 + cos θ )}{2} \vert}{\sqrt{{( \sqrt{{γ}_{L}^{L W}} )}^{2} + {( \sqrt{{γ}_{L}^{-}} )}^{2} + {( \sqrt{{γ}_{L}^{+}} )}^{2}}} .$$

6. The method according to claim 1, wherein the obtaining variation coefficients of the asphalt surface energy parameters corresponding to each of the combinations of the chemical reagents and selecting a group of combinations of the chemical reagents from the combinations of the chemical reagents according to the variation coefficients of the asphalt surface energy parameters corresponding to each of the combinations of the chemical reagents, comprises:
selecting the combinations of the chemical reagents corresponding to the asphalt surface energy parameters of both two kinds of asphalt being not zero from the different chemical reagents, calculating the variation coefficients of the asphalt surface energy parameters of each of the two kinds of asphalt of the respective combinations of the chemical reagents, and selecting the group of combinations of the chemical reagents according to the variation coefficients of the asphalt surface energy parameters of each of the two kinds of asphalt.

7. The method according to claim 6, wherein the obtaining numbers of abnormal values of asphalt surface energy components in the group of combinations of the chemical reagents, comprises:
obtaining the numbers of the abnormal values of asphalt surface energy components of respective combinations of the chemical reagents in the group of combinations of the chemical reagents according to jump degrees of asphalt surface energy components of the combinations of the chemical reagents corresponding to the asphalt surface energy parameters of both the two kinds of asphalt being not zero.

8. The method according to claim 7, wherein the obtaining a target combination of the chemical reagents from the group of combinations of the chemical reagents according to the numbers of the abnormal values, comprises:
taking a combination of the chemical reagents with a least number of the abnormal values of asphalt surface energy components in the group of combinations of the chemical reagents as the target combination of the chemical reagents.

9. The method according to claim 1, wherein each of the combinations of the chemical reagents comprises three kinds of chemical reagents or four kinds of chemical reagents selected from the different chemical reagents.

10. A system for selecting chemical reagents in measurement of asphalt surface energy, comprising: a contact angle obtaining module, an asphalt surface energy parameter obtaining module, a chemical reagent combination group obtaining module and a chemical reagent combination determining module;
wherein the contact angle obtaining module is configured to select different chemical reagents and obtain contact angle values formed between the respective chemical reagents and asphalt slides;
wherein the asphalt surface energy parameter obtaining module is configured to obtain asphalt surface energy parameters corresponding to each of combinations of the chemical reagents according to the contact angle values formed between the respective chemical reagents and the asphalt slides;
wherein the chemical reagent combination group obtaining module is configured to obtain variation coefficients of the asphalt surface energy parameters corresponding to each of the combinations of the chemical reagents and select a group of combinations of the chemical reagents from the combinations of the chemical reagents according to the variation coefficients of the asphalt surface energy parameters corresponding to each of the combinations of the chemical reagents; and
wherein the chemical reagent combination determining module is configured to obtain numbers of abnormal values of asphalt surface energy components in the group of combinations of the chemical reagents and obtain a target combination of the chemical reagents according to the numbers of the abnormal values of asphalt surface energy components; and
wherein the contact angle obtaining module, the asphalt surface energy parameter obtaining module, the chemical reagent combination group obtaining module and the chemical reagent combination determining module are software modules stored in one or more memories and executable by one or more processors coupled to the one or more memories.
